(19) United States
(12) Patent Application Publication
(10) Pub. No.: US 2018/0311439 A1
(43) Pub. Date: Nov. 1, 2018

(54) AN INJECTION DEVICE WITH AN EXPANDABLE CAVITY

(71) Applicant: Sanofi-Aventis Deutschland GMBH, Frankfurt am Main (DE)

(72) Inventors: Stefan Wendland, Frankfurt am Main (DE); Michael Harms, Frankfurt am Main (DE); Zdenek Cerman, Frankfurt am Main (DE)

(21) Appl. No.: 15/778,685

(22) PCT Filed: Nov. 21, 2016
(86) PCT No.: PCT/EP2016/078246

§ 371 Date: May 24, 2018

(30) Foreign Application Priority Data
Nov. 27, 2015 (EP) 15196673.6

(51) Int. Cl. A61M 5/20; A61M 5/19; A61M 5/24

(52) U.S. Cl. CPC A61M 5/2053; A61M 5/19; A61M 2005/2086; A61M 5/2459; A61M 2005/2013; A61M 5/2066

(57) ABSTRACT

An injector device comprises a housing having a distal end and a proximal end; a medicament reservoir; a stopper for expelling a medicament out of the medicament reservoir; a cavity having an expandable volume which is arranged to move the reservoir or the stopper in a distal direction when the cavity is at least partially inflated with a fluid; and a fluid reservoir which is configured to dispense the fluid in to the cavity; wherein the cavity comprises a flexible tube, and the fluid reservoir is configured to dispense the fluid into the flexible tube.

### CROSS REFERENCE TO RELATED APPLICATIONS

The present application is the national stage entry of International Patent Application No. PCT/EP2016/078246, filed on Nov. 12, 2016, and claims priority to Application No. EP 15196673.6, filed in on Nov. 27, 2015, the disclosures of which are incorporated herein by reference.

### TECHNICAL FIELD

The present disclosure relates to an injection device.

### BACKGROUND

Injection devices, such as auto-injectors, are known in the art for dispensing a medicament to an injection site of a user. Such injection devices typically comprise a body and a cap. A needle syringe is located in the body. The cap is removably attached to the body to shield the needle of the needle syringe. To dispense the medicament, the cap is first removed from the body to expose the needle. The needle is then inserted into the body of the user at the injection site to dispense the medicament.

The medicament is typically dispensed using by a piston which moves through a medicament chamber of the syringe to expel the medicament through the needle. Such pistons may be as long as the medicament chamber itself, which causes the injection device to be very long in an initial state. Alternatively, a short piston may typically be actuated by means of a spring, which adds weight to the injection device.

Injection devices which are compact and lightweight offer improved usability and convenience for a user.

### SUMMARY

According to an aspect, an injector device is provided including a housing having a distal end and a proximal end, a medicament reservoir, a stopper for expelling a medicament out of the medicament reservoir, a cavity having an expandable volume which is arranged to move the reservoir or the stopper in a distal direction when the cavity is at least partially inflated with a fluid, and a fluid reservoir which is configured to dispense the fluid into the cavity, wherein the cavity includes a flexible tube, and the fluid reservoir is configured to dispense the fluid into the flexible tube.

The longitudinal extent of the flexible tube when inflated may be greater than the longitudinal extent of the fluid reservoir.

The volume of the fluid reservoir may be greater than the volume of the flexible tube when inflated.

The injector device may include a piston for expelling the fluid from the fluid reservoir into the cavity.

The fluid reservoir may include a first chamber for storing a first medium, and a second chamber for storing a second medium. The first medium and second medium may react if mixed to form a third medium having a greater volume than the total volume of the first and second medium.

The third medium may be expelled into the cavity by fluid pressure resulting from mixing the first medium and the second medium.

The first chamber and the second chamber may be separated by a frangible membrane.

The injector device may include a pointed tip configured to rupture the frangible membrane.

The first chamber and the second chamber may be separated by a valve.

The injector device may include a piston for expelling the first medium from the first chamber into the second chamber.

The second chamber and the flexible tube may be separated by a second frangible membrane.

The second membrane may be configured to be ruptured by the formation of the third medium.

The medicament reservoir may include a needle at a distal end of the reservoir. The cavity may be arranged to move the reservoir in a distal direction and to move the needle out of the distal end of the housing.

The injector device may include a medicament which is retained in the medicament reservoir and is arranged to be expelled out of the medicament reservoir by the stopper.

An auto-injector device may include the injector device and an activation mechanism for activating the dispense mechanism.

According to another aspect, a method of operating an injecting device is provided, including inflating a cavity having an expandable volume with a fluid, moving a medicament reservoir in a distal direction or moving a stopper in a distal direction through the medicament reservoir, when the cavity is at least partially inflated with a fluid.

The terms “drug” or “medicament” which are used interchangeably herein, mean a pharmaceutical formulation that includes at least one pharmaceutically active compound.

The term “drug delivery device” shall be understood to encompass any type of device, system or apparatus designed to immediately dispense a drug to a human or non-human body (veterinary applications are clearly contemplated by the present disclosure). By “immediately dispense” is meant an absence of any necessary intermediate manipulation of the drug by a user between discharge of the drug from the drug delivery device and administration to the human or non-human body. Without limitation, typical examples of drug delivery devices may be found in injection devices, inhalers, and stomach tube feeding systems. Again without limitation, exemplary injection devices may include, e.g., syringes, autoinjectors, injection pen devices and spinal injection systems.

These and other aspects will be apparent from and elucidated with reference to the embodiments described hereinafter.

### BRIEF DESCRIPTION OF THE FIGURES

Exemplary embodiments of the present invention are described with reference to the accompanying drawings, in which:

FIG. 1A is a schematic side view of an injection device according to an exemplary embodiment, with a cap attached to a body of the injection device;

FIG. 1B is a schematic side view of the injection device of FIG. 1A, with the cap removed from the body;

FIG. 2 is a schematic cross-sectional side view of the FIGS. 1A and 1B injection device according to an exemplary embodiment;

FIG. 3 is a schematic cross-sectional side view of the injection device of FIG. 2;

FIG. 4 is a schematic cross-sectional side view of the injection device of FIG. 2;

FIG. 5a is a schematic cross-sectional side view of the FIGS. 1A and 1B injection device according to an exemplary embodiment;

FIG. 5b is a schematic cross-sectional side view of the injection device of FIG. 5a;

FIG. 5c is a schematic cross-sectional side view of the injection device of FIG. 5a.

FIG. 6 is a schematic cross-sectional side view of the FIGS. 1A and 1B injection device according to an exemplary embodiment; and

FIG. 7 is a schematic cross-sectional side view of the FIGS. 1A and 1B injection device according to an exemplary embodiment;

### DETAILED DESCRIPTION

One or more embodiments provide an improved dispense mechanism for a syringe or an auto-injector device, wherein the dispense mechanism includes a flexible tube as a driving element. The flexible tube increases in length when inflated, which can be used to drive the injection of a medicament. The flexible tube can be rolled or folded when deflated, and so provides an injection device which is relatively compact.

A drug delivery device, as described herein, may be configured to inject a medicament into a patient. For example, delivery could be sub-cutaneous, intra-muscular, or intravenous. Such a device could be operated by a patient or care-giver, such as a nurse or physician, and can include various types of safety syringe, pen-injector, or auto-injector. The device can include a cartridge-based system that requires piercing a sealed ampule before use. Volumes of medicament delivered with these various devices can range from about 0.5 ml to about 2 ml. Yet another device can include a large volume device (“LVD”) or patch pump, configured to adhere to a patient's skin for a period of time (e.g., about 5, 15, 30, 60, or 120 minutes) to deliver a “large” volume of medicament (typically about 2 ml to about 5 ml).

In combination with a specific medicament, the presently described devices may also be customized in order to operate within required specifications. For example, the device may be customized to inject a medicament within a certain time period (e.g., about 3 to about 20 seconds for auto-injectors, and about 10 minutes to about 60 minutes for an LVD). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, a drug delivery device will often include a hollow needle ranging from about 25 to about 31 Gauge in size. Common sizes are 27 and 29 Gauge.

The delivery devices described herein can also include one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of an auto-injector may each be activated via an activation mechanism. Such an activation mechanism can include one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

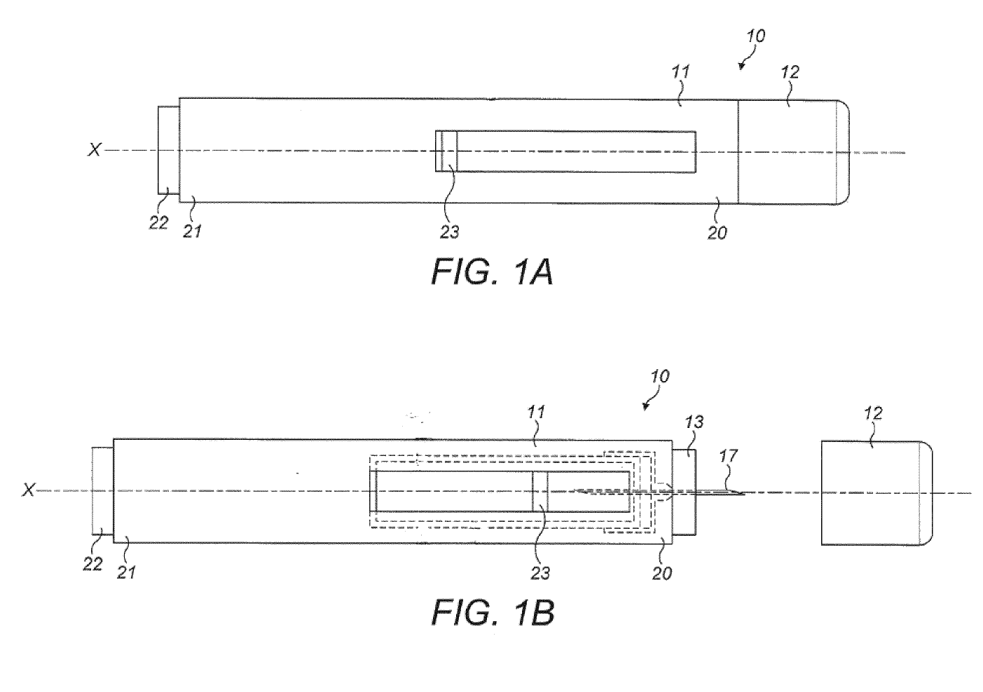

According to some embodiments of the present disclosure, an exemplary drug delivery device 10 is shown in FIGS. 1A & 1B. Device 10, as described above, is configured to inject a medicament into a patient's body. Device 10 includes a housing 11 which typically contains a reservoir containing the medicament to be injected (e.g., a syringe) and the components required to facilitate one or more steps of the delivery process. Device 10 can also include a cap assembly 12 that can be detachably mounted to the housing 11. Typically a user must remove cap 12 from housing 11 before device 10 can be operated.

As shown, housing 11 is substantially cylindrical and has a substantially constant diameter along the longitudinal axis X. The housing 11 has a distal region 20 and a proximal region 21. The term “distal” refers to a location that is relatively closer to a site of injection, and the term “proximal” refers to a location that is relatively further away from the injection site.

Device 10 can also include a needle sleeve 13 coupled to housing 11 to permit movement of sleeve 13 relative to housing 11. For example, sleeve 13 can move in a longitudinal direction parallel to longitudinal axis X. Specifically, movement of sleeve 13 in a proximal direction can permit a needle 17 to extend from distal region 20 of housing 11.

Insertion of needle 17 can occur via several mechanisms. For example, needle 17 may be fixedly located relative to housing 11 and initially be located within an extended needle sleeve 13. Proximal movement of sleeve 13 by placing a distal end of sleeve 13 against a patient's body and moving housing 11 in a distal direction will uncover the distal end of needle 17. Such relative movement allows the distal end of needle 17 to extend into the patient's body. Such insertion is termed “manual” insertion as needle 17 is manually inserted via the patient's manual movement of housing 11 relative to sleeve 13.

Another form of insertion is “automated,” whereby needle 17 moves relative to housing 11. Such insertion can be triggered by movement of sleeve 13 or by another form of activation, such as, for example, a button 22. As shown in FIGS. 1A & 1B, button 22 is located at a proximal end of housing 11. However, in other embodiments, button 22 could be located on a side of housing 11.

Other manual or automated features can include drug injection or needle retraction, or both. Injection is the process by which a bung or piston 23 is moved from a proximal location within a syringe (not shown) to a more distal location within the syringe in order to force a medicament from the syringe through needle 17. In some embodiments, a drive spring (not shown) is under compression before device 10 is activated. A proximal end of the drive spring can be fixed within proximal region 21 of housing 11, and a distal end of the drive spring can be configured to apply a compressive force to a proximal surface of piston 23. Following activation, at least part of the energy stored in the drive spring can be applied to the proximal surface of piston 23. This compressive force can act on piston 23 to move it in a distal direction. Such distal movement acts to compress the liquid medicament within the syringe, forcing it out of needle 17.

Following injection, needle 17 can be retracted within sleeve 13 or housing 11. Retraction can occur when sleeve 13 moves distally as a user removes device 10 from a patient's body. This can occur as needle 17 remains fixedly located relative to housing 11. Once a distal end of sleeve 13 has moved past a distal end of needle 17, and needle 17 is covered, sleeve 13 can be locked. Such locking can include locking any proximal movement of sleeve 13 relative to housing 11.

Another form of needle retraction can occur if needle 17 is moved relative to housing 11. Such movement can occur if the syringe within housing 11 is moved in a proximal direction relative to housing 11. This proximal movement can be achieved by using a retraction spring (not shown), located in distal region 20. A compressed retraction spring, when activated, can supply sufficient force to the syringe to move it in a proximal direction. Following sufficient retraction, any relative movement between needle 17 and housing 11 can be locked with a locking mechanism. In addition, button 22 or other components of device 10 can be locked as required.

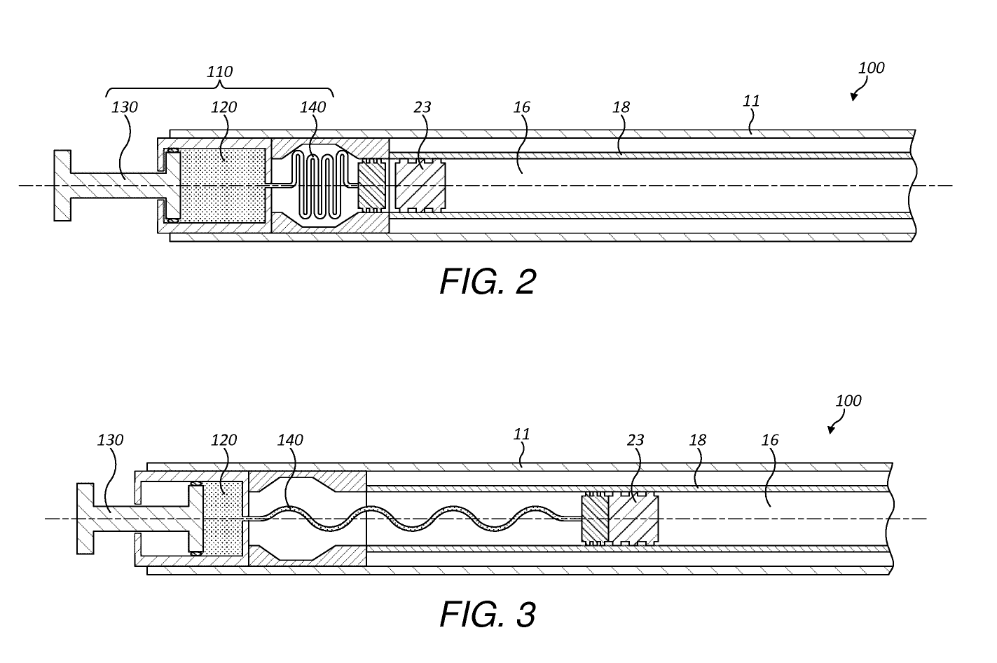

With reference to FIG. 2, an injection device 100 according to a first embodiment is shown. The injection device 100 comprises a syringe 18 containing liquid medicament 16, substantially as described with respect to FIGS. 1a and 1b, and a dispense mechanism 110 configured to displace the rubber stopper 23 of the syringe 18 toward the proximal end of the medicament chamber.

The dispense mechanism 110 comprises a fluid reservoir 120, a piston 130 and a flexible tube 140. The piston 130 is disposed within the fluid reservoir 120, and the flexible tube 140 is coupled between the fluid reservoir 120 and the rubber stopper 23 of the syringe 18. The flexible tube 140 is connected with the fluid reservoir 120 such that a fluid or gas is able to move between the two and is mechanically coupled to the rubber stopper 23 of the syringe 18 such that a pushing force is exerted on the rubber stopper 23 when the flexible tube 140 expands.

The piston 130 of the dispense mechanism 110 is configured to move through the fluid reservoir 120 and push at least a portion of a fluid contained therein into the flexible tube 140. The piston 130 comprises a piston stopper disposed at the proximal end of the piston 130, a piston head at the proximal end of the piston 130 and a shaft connecting the piston stopper with the piston head. The piston stopper is disposed in the fluid reservoir 120 and is arranged to push the fluid from the fluid reservoir 120 into the flexible tube 140. The piston head has a broad cross section which is disposed at a proximal end of the housing 11, arranged to be pushed by a user in order to dispense the medicament 16. The shaft has a reduced cross-section in comparison with the piston stopper and the piston head, and connects the two parts through an opening in the proximal end of the fluid reservoir 120 and an opening in the proximal end of the housing 11.

The piston 130 can be pushed axially into the housing 11 in order to move the piston stopper axially toward the proximal end of the fluid reservoir 120, ejecting the fluid from the fluid reservoir 120 into the flexible tube 140. The flexible tube 140 is inflated by the fluid entering from the fluid reservoir 120 and expands, which causes the flexible tube 140 to exert a pushing force of the rubber stopper 23 of the syringe 18. The movement of the piston 130 is therefore translated into a displacement of the rubber stopper 23 which causes a medicament 16 to be dispensed through the needle of the syringe 18.

FIG. 2 shows the syringe 18 of the first embodiment in an initial state wherein the plunger is not pushed and substantially all of the fluid is disposed within the fluid reservoir 120. The flexible tube 140 can be folded or rolled when empty and therefore has a reduced length when empty compared to the length when inflated. The length of the dispense mechanism 110 can therefore be reduced and the syringe 18 can be provided in a more compact package.

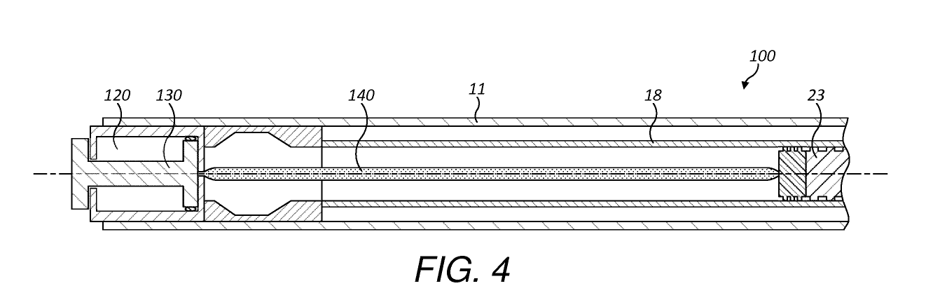

FIG. 3 and FIG. 4 show the syringe 18 of the first embodiment with the piston 130 positioned respectively halfway and fully toward the distal end of the fluid reservoir 120. As the piston 130 is moved axially through the fluid reservoir 120 the fluid contained therein is forced into the flexible tube 140. The flexible tube 140 is inflated by the fluid entering from the fluid reservoir 120 and is forced to unfold or unroll by the internal pressure exerted by the fluid. The expansion of the flexible tube 140 exerts an axial force of the rubber stopper 23 of the syringe 18, and causes the rubber stopper 23 to move axially toward a distal end of the medicament chamber.

When the piston 130 reaches the final position, as shown in FIG. 4, substantially all of the fluid has been deployed into the flexible tube 140, which is fully inflated and maximally extended as a result. The fully extended flexible tube 140 forces the rubber stopper 23 of the syringe 18 to the distal end of the medicament chamber, such that substantially all of the medicament 16 is dispensed through the needle as the flexible tube 140 is inflated.

The cross-section of the flexible tube 140 is smaller than the cross section of the fluid reservoir 120. As a result, the fluid reservoir 120 is capable of retaining at least the volume of fluid required to fully inflate the flexible tube 140 with a smaller length than the flexible tube 140. The combined length of the extended piston 130 and the empty flexible tube 140 in an initial state is shorter than that of a conventional piston for a similar volume syringe 18. The dispensing mechanism according to the first embodiment therefore provides for an improved syringe 18 which is more compact.

The syringe 18 may be configured to move slideably along the length of the housing 11. The expansion of the flexible tube 140 exerts an axial force on the syringe 18. The expansion of the flexible tube 140 may cause the syringe 18 to move in a distal direction. Movement of the syringe 18 may cause the needle 17 to move in a distal direction. The needle 17 may initially positioned within a distal end of the housing 11. The expansion of the flexible tube 140 may cause the needle 17 to move out of the distal end of the housing 11.

The piston 130 may be pushed in a distal direction to move the syringe 18 in a distal direction and move the needle 17 out of the distal end of the housing 11. The syringe 18 stops at the distal end of the housing 11. Further movement of the piston 130 causes the rubber stopper 23 to move through the medicament chamber.

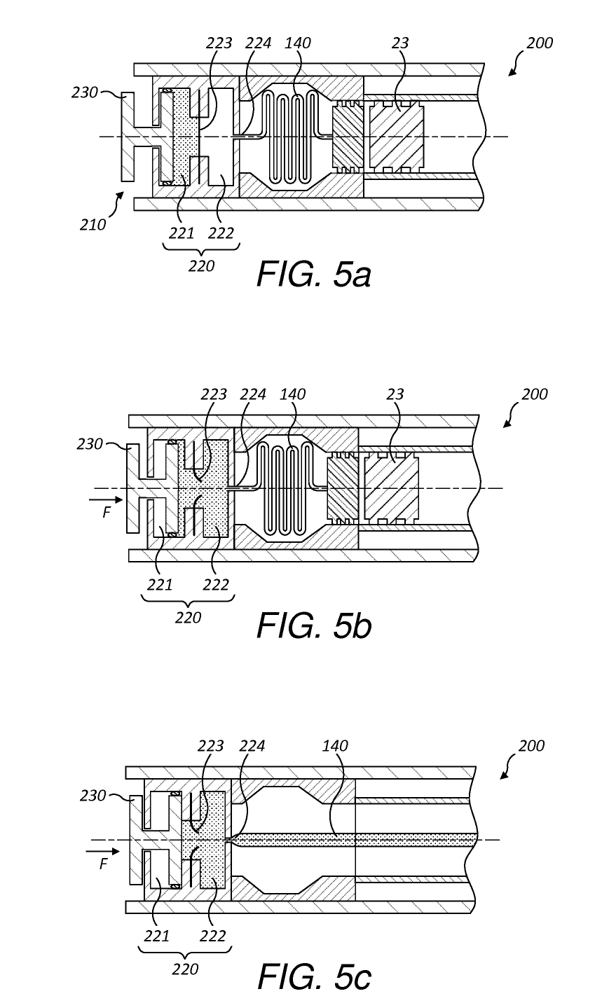

With reference to FIGS. 5a, 5b and 5c, an injector device 200 according to a second embodiment is described. Elements of the embodiment which are not described are substantially the same as those of the first embodiment.

A dispense mechanism 210 comprises a fluid reservoir 220 including a first proximal chamber 221 and a second distal chamber 222. A piston 230 comprises a piston stopper disposed within the first chamber 221, which is arranged to move from a proximal end towards a distal end of the first chamber 221. The first chamber 221 and the second chamber 222 are separated by a frangible membrane 223 or, alternatively, by a valve, which allows a fluid to pass from the first chamber 221 into the second chamber 222 under pressure. The fluid reservoir 220 includes a narrow portion having a reduced cross-section over which the membrane 223 or valve is placed.

FIG. 5a shows an initial state of the dispense mechanism 210 according to the second embodiment. The first proximal chamber 221 contains a first medium and the second distal chamber 222 contains a second medium. The first medium and second medium, when mixed, react to produce a third medium which has a greater volume than the initial volume of the first medium and the second medium. Each of the first, second and third medium may be a liquid or a gas.

The piston 230 can be pushed axially into the housing 11 in order to move the piston stopper axially toward the distal end of the first chamber 221, which forces the first medium through the membrane 223 from the first chamber 221 into the second chamber 222 and mixes the first medium with the second medium.

FIG. 5b shows an intermediate state of the dispense mechanism 210 according to the second embodiment. There is a reaction between the first medium and the second medium which results in the third medium being produced within the second chamber 222. There is a second frangible membrane 224 disposed between the fluid reservoir 220 and the flexible tube 140. The volume of the third medium has a greater volume than that of the second chamber and, as a result, at least a portion of the third medium is forced out of the fluid reservoir 220 through the second membrane 224 into the flexible tube 140.

FIG. 5c shows a final state of the dispense mechanism 210 according to the second embodiment. The flexible tube 140 is inflated by the third medium entering from the fluid reservoir 220 and is forced to unfold or unroll by the internal pressure exerted by the medium. The expansion of the flexible tube 140 exerts an axial force of the rubber stopper 23 of the syringe 18, and causes the rubber stopper 23 to move axially toward a distal end of the medicament chamber.

When the piston 130 is in the final position, as shown in FIG. 5c, substantially all of the first medium has been forced into the second distal chamber 222 and mixed with the second medium. As such, the maximum volume of the third medium is produced and the flexible tube 140 becomes fully inflated and maximally extended as a result. The fully extended flexible tube 140 forces the rubber stopper 23 of the syringe 18 to the distal end of the medicament chamber, such that substantially all of the medicament 16 is dispensed through the needle as the flexible tube 140 is inflated.

Whereas the injector device 100 according to the first embodiment dispenses all of medicament 16 only once the piston 130 reaches the final position at the distal end of the fluid reservoir 120, the injector device 200 of the second embodiment may be configured such that the dispense process will be completed as long as the first fluid and second fluid are mixed to some extent. The injector device 200 according to the second embodiment will therefore deliver all of the medicament 16, provided the frangible membrane 223 has been burst. The second embodiment is therefore particularly suitable for use in an auto-injector device.

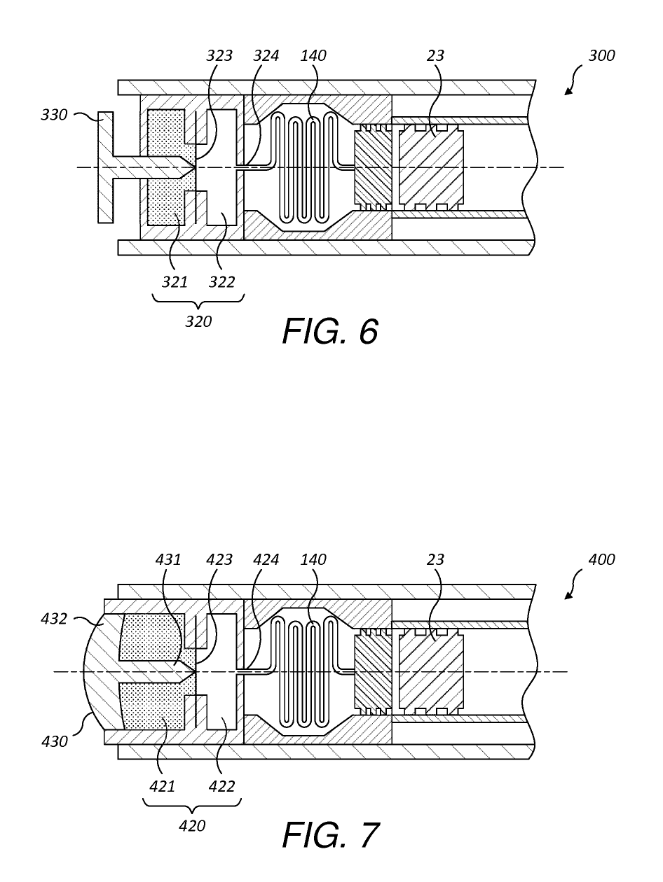

With reference to FIG. 6, an injector device 300 according to a third embodiment is described. A dispense mechanism 310 comprises a fluid reservoir 320 including a first proximal chamber 321 and a second distal chamber 322. The first chamber 321 and the second chamber 322 are separated by a frangible membrane 323 which covers a narrow portion of the fluid reservoir 320 having a reduced cross-section. The dispense mechanism 310 further comprises a piercing element 330 arranged to rupture the frangible membrane 323.

The piercing element 330 is shaped, at the proximal head, like the piston head described in the first and second embodiments, so as to be pushed in a distal direction by the user. The distal end of the piercing element 330 is formed with a pointed tip which is capable of rupturing the frangible membrane 323 when the piercing element 330 is brought into contact with the frangible membrane 323 and pressure is applied.

FIG. 6 shows an initial state of the dispense mechanism 310 according to the third embodiment. The first proximal chamber 321 contains a first medium and the second distal chamber 322 contains a second medium. The first medium and second medium, when mixed, react to produce a third medium which has a greater volume than the initial volume of the first medium and the second medium. Each of the first, second and third medium may be a liquid or a gas.

The piercing element 330 can be pushed axially into the housing 11 in order to move the pointed tip axially toward, and subsequently through, the frangible membrane 323. When the frangible membrane 323 is ruptured by the piercing element 330 the first medium can flow from the first chamber 321 into the second chamber 322 and mix with the second medium therein.

The reaction between the first medium and the second medium results in the third medium being produced within the second chamber 322, and, as the volume of the third medium has a greater volume than that of the second chamber 322, at least a portion of the third medium is forced out of the fluid reservoir 320. The third medium is forced through the second membrane 324 into the flexible tube 140, which is forced to unfold or unroll by the internal pressure exerted by the third medium. The expansion of the flexible tube 140 exerts an axial force of the rubber stopper 23 of the syringe, and causes the rubber stopper 23 to move axially toward a distal end of the medicament chamber.

The injector device 300 of the third embodiment is configured such that the dispense process will be completed as long as the first fluid and second fluid are mixed to some extent. The injector device 300 according to the third embodiment will therefore deliver all of the medicament 16, provided that the piercing element 330 has ruptured the frangible membrane 323. The third embodiment is therefore particularly suitable for use in an auto-injector device.

With reference to FIG. 7, an injector device 400 according to a fourth embodiment is described. A dispense mechanism 410 comprises a fluid reservoir 420 including a first proximal chamber 421 and a second distal chamber 422. The first chamber 421 and the second chamber 422 are separated by a frangible membrane 423 which covers a narrow portion of the fluid reservoir 420 having a reduced cross-section. The dispense mechanism 410 further comprises a piercing element 430 arranged to rupture the frangible membrane 423.

The distal end of the piercing element 430 is formed with a pointed tip 431 which is capable of rupturing the frangible membrane 423 when the piercing element 430 is brought into contact with the frangible membrane 423 and pressure is applied. The proximal end of the piercing element 430 comprises an elastic membrane 432 arranged to cover and seal the proximal end of the first proximal chamber 421. The elastic membrane 432 may lie flat across the opening in the first proximal chamber 421 or may initially be biased to curve outwards.

FIG. 7 shows an initial state of the dispense mechanism 410 according to the fourth embodiment. The first proximal chamber 421 contains a first medium and the second distal chamber 422 contains a second medium. The first medium and second medium, when mixed, react to produce a third medium which has a greater volume than the initial volume of the first medium and the second medium. Each of the first, second and third medium may be a liquid or a gas.

The piercing element 430 can be pushed axially into the housing 11 in order to move the pointed tip 431 axially toward, and subsequently through, the frangible membrane 423. When the frangible membrane 423 is ruptured by the piercing element 430 the first medium can flow from the first chamber 421 into the second chamber 422 and mix with the second medium therein. The elastic membrane 432 flexes inwards with respect to the first proximal chamber 421 when pushed, which allows the pointed tip 431 to contact and pierce the frangible membrane 423. The flexion of the elastic membrane 432 reduces the volume of the first proximal chamber 421 and urges the first medium through the ruptured frangible membrane 423 into the second distal chamber 422.

The reaction between the first medium and the second medium results in the third medium being produced within the second chamber 422, and, as the volume of the third medium has a greater volume than that of the second chamber 422, at least a portion of the third medium is forced out of the fluid reservoir 420. The third medium is forced through the second membrane 424 into the flexible tube 140, which is forced to unfold or unroll by the internal pressure exerted by the third medium. The expansion of the flexible tube 140 exerts an axial force of the rubber stopper 23 of the syringe, and causes the rubber stopper 23 to move axially toward a distal end of the medicament chamber.

The injector device 400 of the fourth embodiment is configured such that the dispense process will be completed as long as the first fluid and second fluid are mixed to some extent. The injector device 400 according to the third embodiment will therefore deliver all of the medicament 16, provided that the piercing element 430 has ruptured the frangible membrane 423. The fourth embodiment is therefore particularly suitable for use in an auto-injector device.

Although a few embodiments have been shown and described, it will be appreciated by those skilled in the art that changes may be made in these embodiments without departing from the disclosure, the scope of which is defined in the appended claims. Various components of different embodiments may be combined where the principles underlying the embodiments are compatible.

For example, the injector device of any embodiment may be formed independently to provide a dispense mechanism for a compact syringe device, or may be utilised as part of an auto-injector device in which the dispense mechanism is actuated by an automatic activation mechanism. The device may be a needle-less device, which is configured to squirt a fine jet of liquid medicament at sufficiently high pressure to penetrate the skin at the injection site.

The flexible tube of any embodiment may have any suitable shape, for example, a cylindrical form or a flat ribbon form. In some embodiments, the device may include a plurality of tubes. The flexible tube may be inflated by any suitable means, for example, by a compressed gas source or by heat expansion of a gas.

Alternatively, some embodiments may operate without a flexible tube, wherein the stopper is pushed through the syringe by the inflation of a cavity having an expandable volume at the proximal end of the syringe. An expansion chamber may be formed in a proximal portion of the syringe which is separated from the medicament chamber by the stopper. The expansion chamber may be inflated by a fluid from, for example, a fluid reservoir as described in any embodiment. The fluid may be forced from the fluid reservoir into the expansion chamber by a chemical reaction or by a mechanical operation to reduce the volume of the fluid reservoir. The fluid reservoir may be external to the device, for example, it may be connected to the expansion chamber only by a fluid conduit. Alternatively, the expansion chamber may be caused to inflate by the expansion of a chemical medium within the expansion chamber.

Embodiments of the dispense mechanism may include a single chemical medium only, which is configured to expand to a greater volume when brought into contact with a catalyst or an activation surface. The piercing element described in each of the third and fourth embodiments may comprise an activation surface to cause the expansion of a chemical medium in the distal chamber. The proximal chamber remains empty and the device is activated when the pointed tip and activation surface of the piercing element is pushed into the distal chamber. In some embodiments, the distal chamber may be arranged adjacent to the stopper of the syringe and may form an expansion chamber. The chemical expansion of the medium within the expansion chamber increases the volume of the chamber by forcing the stopper through the syringe in a distal direction.

The fluid reservoir of any embodiment may be open to the flexible tube, that is, the second membrane described with respect to the second embodiment may be optional. The flexible tube is initially rolled or folded, which is sufficient to retain the fluid in the fluid reservoir until sufficient pressure is applied.

The piston of the first and second embodiments may have any suitable form, for example, the piston may be a cylindrical button without a broad piston head, or may be part of a level mechanism. The piston may be driven through the fluid reservoir manually, or otherwise by an electric motor, by a compressed gas release or by any other dispense mechanism suitable for use with an auto-injector device.

The terms “drug” or “medicament” are used herein to describe one or more pharmaceutically active compounds. As described below, a drug or medicament can include at least one small or large molecule, or combinations thereof, in various types of formulations, for the treatment of one or more diseases. Exemplary pharmaceutically active compounds may include small molecules; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more of these drugs are also contemplated.

The term “drug delivery device” shall encompass any type of device or system configured to dispense a drug into a human or animal body. Without limitation, a drug delivery device may be an injection device (e.g., syringe, pen injector, auto injector, large-volume device, pump, perfusion system, or other device configured for intraocular, subcutaneous, intramuscular, or intravascular delivery), skin patch (e.g., osmotic, chemical, micro-needle), inhaler (e.g., nasal or pulmonary), implantable (e.g., coated stent, capsule), or feeding systems for the gastro-intestinal tract. The presently described drugs may be particularly useful with injection devices that include a needle, e.g., a small gauge needle.

The drug or medicament may be contained in a primary package or “drug container” adapted for use with a drug delivery device. The drug container may be, e.g., a cartridge, syringe, reservoir, or other vessel configured to provide a suitable chamber for storage (e.g., short- or long-term storage) of one or more pharmaceutically active compounds. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of a drug formulation (e.g., a drug and a diluent, or two different types of drugs) separately, one in each chamber. In such instances, the two chambers of the dual-chamber cartridge may be configured to allow mixing between the two or more components of the drug or medicament prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drug delivery devices and drugs described herein can be used for the treatment and/or prophylaxis of many different types of disorders. Exemplary disorders include, e.g., diabetes mellitus or complications associated with diabetes mellitus such as diabetic retinopathy, thromboembolism disorders such as deep vein or pulmonary thromboembolism. Further exemplary disorders are acute coronary syndrome (ACS), angina, myocardial infarction, cancer, macular degeneration, inflammation, hay fever, atherosclerosis and/or rheumatoid arthritis.

Exemplary drugs for the treatment and/or prophylaxis of diabetes mellitus or complications associated with diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the term “derivative” refers to any substance which is sufficiently structurally similar to the original substance so as to have substantially similar functionality or activity (e.g., therapeutic effectiveness).

Exemplary insulin analogues are Gly(A21), Arg(B31), Arg(B32) human insulin (insulin glargine); Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Exemplary insulin derivatives are, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin; B29-N—(N-lithocholyl-gamma-glutamyl)-des(B30) human insulin; B29-N-(ω-carboxyheptadecanoyl)-des(B30) human insulin and B29-N-(ω-carboxyhepta¬decanoyl) human insulin. Exemplary GLP-1, GLP-1 analogues and GLP-1 receptor agonists are, for example: Lixisenatide/AVE0010/ZP10/Lyxumia, Exenatide/Exendin-4/Byetta/Bydureon/ITCA 650/AC-2993 (a 39 amino acid peptide which is produced by the salivary glands of the Gila monster), Liraglutide/Victoza, Semaglutide, Taspoglutide, Syncria/Albiglutide, Dulaglutide, rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An exemplary oligonucleotide is, for example: mipomersen/Kynamro, a cholesterol-reducing antisense therapeutic for the treatment of familial hypercholesterolemia.

Exemplary DPP4 inhibitors are Vildagliptin, Sitagliptin, Denagliptin, Saxagliptin, Berberine.

Exemplary hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Exemplary polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20/Synvisc, a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region.

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Exemplary antibodies are anti PCSK-9 mAb (e.g., Alirocumab), anti IL-6 mAb (e.g., Sarilumab), and anti IL-4 mAb (e.g., Dupilumab).

The compounds described herein may be used in pharmaceutical formulations comprising (a) the compound(s) or pharmaceutically acceptable salts thereof, and (b) a pharmaceutically acceptable carrier. The compounds may also be used in pharmaceutical formulations that include one or more other active pharmaceutical ingredients or in pharmaceutical formulations in which the present compound or a pharmaceutically acceptable salt thereof is the only active ingredient. Accordingly, the pharmaceutical formulations of the present disclosure encompass any formulation made by admixing a compound described herein and a pharmaceutically acceptable carrier.

Pharmaceutically acceptable salts of any drug described herein are also contemplated for use in drug delivery devices. Pharmaceutically acceptable salts are for example acid addition salts and basic salts. Acid addition salts are e.g. HCl or HBr salts. Basic salts are e.g. salts having a cation selected from an alkali or alkaline earth metal, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are known to those of skill in the arts.

Pharmaceutically acceptable solvates are for example hydrates or alkanolates such as methanolates or ethanolates.

Those of skill in the art will understand that modifications (additions and/or removals) of various components of the substances, formulations, apparatuses, methods, systems and embodiments described herein may be made without departing from the full scope of the present disclosure, which encompass such modifications and any and all equivalents thereof.

1. An injector device comprising:
a housing having a distal end and a proximal end;
a medicament reservoir;
a stopper for expelling a medicament out of the medicament reservoir;
a cavity having an expandable volume which is arranged to move the reservoir or the stopper in a distal direction when the cavity is at least partially inflated with a fluid; and
a fluid reservoir which is configured to dispense the fluid in to the cavity;
wherein the cavity comprises a flexible tube, and the fluid reservoir is configured to dispense the fluid into the flexible tube.

2. The injector device of claim 1, wherein a longitudinal extent of the flexible tube when inflated is greater than a longitudinal extent of the fluid reservoir.

3. The injector device of claim 1, wherein a volume of the fluid reservoir is greater than a volume of the flexible tube when inflated.

4. The injector device of claim 1, further comprising a piston for expelling the fluid from the fluid reservoir into the cavity.

5. The injector device of claim 1, wherein the fluid reservoir comprises:
a first chamber for storing a first medium; and
a second chamber for storing a second medium;
wherein the first medium and second medium react if mixed to form a third medium having a greater volume than the total volume of the first and second mediums, and
wherein the third medium is expelled into the cavity by fluid pressure resulting from mixing the first medium and the second medium.

6. The injector device of claim 5, wherein the first chamber and the second chamber are separated by a frangible membrane.

7. The injector device of claim 6, further comprising a pointed tip arranged to rupture the frangible membrane.

8. The injector device of claim 5, wherein the first chamber and the second chamber are separated by a valve.

9. The injector device of claim 5, further comprising a piston for expelling the first medium from the first chamber into the second chamber.

10. The injector device of claim 5, wherein the second chamber and the flexible tube are separated by a second frangible membrane, and the second membrane is configured to be ruptured by the formation of the third medium.

11. The injector device of claim 1, wherein the medicament reservoir comprises a needle at a distal end of the reservoir; and
wherein the cavity is arranged to move the reservoir in a distal direction and to move the needle out of the distal end of the housing.

12. The injector device of claim 1, further comprising a medicament which is retained in the medicament reservoir and is arranged to be expelled out of the medicament reservoir by the stopper.

13. An auto-injector device, comprising:
an injector device according to claim 1; and
an activation mechanism for activating dispensing of the fluid by the fluid mechanism.

14. A method of operating an injecting device, the method comprising:
inflating a cavity comprising a flexible tube and having an expandable volume with a fluid by dispensing the fluid from a fluid reservoir into the flexible tube; and
moving a medicament reservoir in a distal direction or moving a stopper in a distal direction through the medicament reservoir when the cavity is at least partially inflated with a fluid.
